(19) United States
(12) Patent Application Publication
(10) Pub. No.: US 2023/0203067 A1
(43) Pub. Date: Jun. 29, 2023

(54) PRODUCTION PROCESS FOR AMINO ALCOHOLATES

(71) Applicant: Covestro Deutschland AG, Leverkusen (DE)

(72) Inventors: Frank Richter, Leverkusen (DE); Michael Traving, Leverkusen (DE); Joerg Kolle, Kerpen-Horrem (DE); Klaus Lorenz, Dormagen (DE)

(21) Appl. No.: 17/998,831

(22) PCT Filed: Jun. 4, 2021
(86) PCT No.: PCT/EP2021/065019

§ 371 Date: Nov. 15, 2022

(30) Foreign Application Priority Data
Jun. 8, 2020 (EP) 20178639.9

(51) Int. Cl. C07F 7/22; C07C 213/06; C08G 18/24; B01J 31/02; C09D 175/04; C08G 18/73

(52) U.S. Cl. CPC C07F 7/2224; C07C 213/06; C08G 18/24; B01J 31/0211; C09D 175/04; C08G 18/73

(57) ABSTRACT

The invention relates to a method for producing at least one metal amino alcoholate, at least comprising the following steps (A) providing at least one amino alcohol, (B) adding at least one basic compound to the at least one amino alcohol provided in step (A) in order to obtain at least one corresponding amino alcoholate, and (C) adding at least one metal halogenide to the mixture obtained in step (C) in order to obtain a corresponding metal amino alcoholate, wherein in step (C) the at least one metal halogenide is added as a solution in a protic solvent at a concentration of 2.0 to 35.0 wt. %; a solution containing at least one metal alcoholate obtained in this manner; the use of the solution to produce a composition; a corresponding composition; the use of said composition to produce single- or multi-layer paint structures; a method for single- or multi-layer coating of a substrate with a paint structure; and a substrate coated in this manner.

The invention relates to a method for preparing at least one metal amino alkoxide, at least comprising the following steps (A) providing at least one amino alcohol,

(B) adding at least one basic compound to the at least one amino alcohol provided in step (A) in order to obtain at least one corresponding amino alkoxide, and (C) adding at least one metal halide to the mixture obtained in step (C) in order to obtain the corresponding at least one metal amino alkoxide, wherein in step (C) the at least one metal halide is added as a solution in a protic solvent at a concentration of 2.0 to 35.0% by weight. The present invention further relates to a solution comprising at least one correspondingly prepared metal alkoxide, to the use of the solution for producing a composition, to a corresponding composition, to the use of said composition for producing single- or multi-layered coating systems, to a method for single- or multi-layered coating of a substrate with a coating system, and to a correspondingly coated substrate.

Methods for preparing metal amino alkoxides, in particular Sn(II) or Sn(IV) alkoxides, and use thereof as thermolatent catalysts in the production of polyurethane coatings are known per se to those skilled in the art.

DE 10 2010 012 237 A discloses a method for preparing tin(II) or tin(IV) alkoxides by reacting elemental tin powder with alcohols in the absence of oxygen and water in order to obtain the corresponding tin(II) alkoxides. If this reaction is carried out in the presence of oxygen and water, the corresponding tin(IV) compounds are obtained.

WO 2014/131750 discloses a method for preparing tin(II) or tin(IV) alkoxides by reacting elemental tin with the appropriate alcohols in the presence of certain activating reagents, for example DMF, DMSO, sulfolane, amine N-oxides or pyridine N-oxide.

WO 09/132784 also discloses tin(IV) catalysts for preparing polyisocyanate polyaddition compounds. These tin(IV) compounds are obtained by oxidizing corresponding tin(II) compounds or by reacting tin(IV) halides with amino alcohols.

WO 2011/051465 discloses a method for preparing metal amino alkoxides by reacting metal halides with alkali metal amino alkoxides in the presence of a base.

The document WO 2011/051247 discloses tin(IV) amino alkoxides as catalysts for producing polyurethane coatings.

Appropriate metal compounds, especially Sn-containing compounds, are used as latent, particularly thermolatent catalysts for producing single-layered or multi-layered coating systems. For this purpose, the catalysts are present in non-polar solvents, for example butyl acetate. These solutions often have a slight yellow discoloration per se, which may intensify on storage and/or particularly when in contact with polyisocyanate-based paint hardeners. Corresponding preparations are often unsuitable for high-quality applications, for example automotive coatings.

The object of the present invention was therefore to provide a method for preparing metal amino alkoxides, in particular tin(IV) amino alkoxides, which makes it possible to obtain the corresponding compounds in high yield. In particular, the metal amino alkoxide prepared according to the invention, in particular tin(IV) amino alkoxide, should be provided, for example for use as a thermolatent catalyst, and on the one hand should not exhibit any discoloration after preparation, in particular no yellow or orange discoloration, and furthermore should not form any discoloration even after storage, in particular no yellow or orange discoloration, so that said compound and a corresponding solution can be obtained efficiently and with consistently high quality, so that they are also reliably suitable for qualitatively demanding applications, such as automotive coatings.

These objects are achieved by the method according to the invention for preparing at least one metal amino alkoxide, at least comprising the following steps:
(A) providing at least one amino alcohol,
(B) adding at least one basic compound to the at least one amino alcohol provided in step (A) in order to obtain at least one corresponding amino alkoxide, and
(C) adding at least one metal halide to the mixture obtained in step (B) in order to obtain the corresponding at least one metal amino alkoxide,
   wherein in step (C) the at least one metal halide is added as a solution in a protic solvent at a concentration of 2.0 to 35.0% by weight.

The objects are also achieved by the solution according to the invention comprising at least one metal amino alkoxide, prepared by the method according to the invention, by the use of the solution according to the invention for producing a composition comprising the at least one metal amino alkoxide, at least one solvent selected from the group consisting of esters, preferably butyl acetate, in particular n-butyl acetate, ethyl acetate, ethylene glycol diacetate, 2-methoxypropyl acetate or mixtures thereof, aromatic solvents, preferably benzene, toluene, xylenes or mixtures thereof, lactones, preferably butyrolactone or mixtures thereof, carbonates, preferably diethyl carbonate, propylene carbonate, ethylene carbonate or mixtures thereof, at least one polyisocyanate and at least one NCO-reactive compound, by the composition according to the invention comprising at least one polyisocyanate, at least one NCO-reactive compound and a solution according to the invention, by the use according to the invention of said composition for producing single- or multi-layered coating systems, by the method according to the invention for single- or multi-layered coating of a substrate with a coating system by applying a corresponding composition to the substrate, and by the substrate according to the invention, coated with a single- or multi-layered coating system comprising a composition according to the invention and/or obtainable by the method according to the invention, in particular where the substrate is a chassis, preferably of a vehicle, especially of a land vehicle, aircraft or watercraft, or a part thereof.

The individual method steps of the method according to the invention are described in detail below.

Step (A) of the method according to the invention comprises providing the at least one amino alcohol.

In general, any amino alcohol known to those skilled in the art can be provided in step (A) of the method of the invention. In the context of the present invention, the term “amino alcohol” signifies an organic molecule which has at least one amino function and at least one hydroxyl function. In a preferred embodiment of the present invention, the at least one amino alcohol provided in step comprises at least one amino function, preferably a secondary or tertiary, particularly preferably a tertiary, amino function and at least two, preferably two, hydroxyl functions.

More preferably, the at least one amino alcohol provided in step (A) of the method according to the invention conforms to the general formula (I) or (II)

HO—X-D-Y—OH (I)

HO—X-D-Z-D-Y—OH (II),

where:

D is —O—, —S— or —N(R1)-, wherein at least one D present is —N(R1)-,

wherein R1 is a saturated or unsaturated, linear or branched, aliphatic or cycloaliphatic radical or an optionally substituted aromatic or araliphatic radical having up to 20 carbon atoms, which may optionally comprise heteroatoms from the group of oxygen, sulfur and nitrogen, or is hydrogen,
X, Y and Z are identical or different radicals selected from alkylene radicals of the formulae —C(R2)(R3)-, —C(R2)(R3)-C(R4)(R5)- or —C(R2)(R3)-C(R4)(R5)-C(R6)(R7)- or ortho-arylene radicals
of the formulae

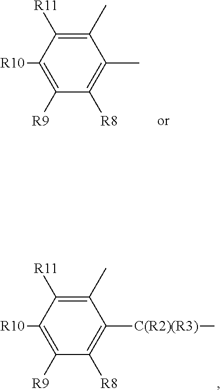

wherein R2 to R11 are each independently saturated or unsaturated, linear or branched, aliphatic or cycloaliphatic or optionally substituted aromatic or araliphatic radicals having up to 20 carbon atoms, which may optionally comprise heteroatoms from the series oxygen, sulfur and nitrogen, or are hydrogen;

X, Y and Z are preferably the alkenylene radicals —C(R2)(R3)-, —C(R2)(R3)-C(R4)(R5)- or the ortho-arylene radical

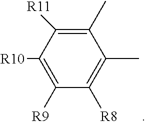

R2 to R7 are preferably hydrogen or alkyl, aralkyl, alkaryl or aryl radicals having up to 20 carbon atoms, particularly preferably hydrogen or alkyl, aralkyl, alkaryl or aryl radicals having up to 8 carbon atoms, very particularly preferably hydrogen or alkyl radicals having up to 8 carbon atoms, even further preferably hydrogen or methyl.

R8 to R11 are preferably hydrogen or alkyl radicals having up to 8 carbon atoms, particularly preferably hydrogen or methyl.

The units HO—X and HO—Y are preferably —CH₂CH₂OH, —CH₂CH(Me)OH, —CH(Me)CH₂OH, —CH₂C(Me)₂OH, —C(Me)₂CH₂OH or —CH₂C(═O)OH.

The unit HO—X-D-Y—OH is preferably: HN[CH₂CH₂OH]₂, HN[CH₂CH(Me)OH]₂, HN[CH₂CH(Me)OH][CH(Me)CH₂OH], HN[CH₂C(Me)₂OH]₂, HN[CH₂C(Me)₂OH][C(Me)₂CH₂OH], HN[CH₂C(═O)OH]₂, MeN[CH₂CH₂OH]₂, MeN[CH₂CH(Me)OH]₂, MeN[CH₂CH(Me)OH][CH(Me)CH₂OH], MeN[CH₂C(Me)₂OH]₂, MeN[CH₂C(Me)₂OH] [C(Me)₂CH₂OH], MeN[CH₂C(═O)OH]₂, EtN[CH₂CH₂OH]₂, EtN[CH₂CH(Me)OH]₂, EtN[CH₂CH(Me)OH] [CH(Me)CH₂OH], EtN[CH₂C(Me)₂OH]₂, EtN[CH₂C(Me)₂OH] [C(Me)₂CH₂OH], EtN[CH₂C(═O)OH]₂, PrN[CH₂CH₂OH]₂, PrN[CH₂CH(Me)OH]₂, PrN [CH₂CH(Me)OH] [CH(Me)CH₂OH], PrN[CH₂C(Me)₂OH]₂, PrN[CH₂C(Me)₂OH] [C(Me)₂CH₂OH], PrN[CH₂C(═O)OH]₂, BuN[CH₂CH₂OH]₂, BuN[CH₂CH(Me)OH]₂, BuN[CH₂CH(Me)OH][CH(Me)CH₂OH], PenN[CH₂C(Me)₂OH]₂, PenN[CH₂C(Me)₂OH] [C(Me)₂CH₂OH], PenN[CH₂C(═O)OH]₂, PenN[CH₂CH₂OH]₂, PenN[CH₂CH(Me)OH]₂, PenN [CH₂CH(Me)OH] [CH(Me)CH₂OH], PenN [CH₂C(Me)₂OH]₂, PenN[CH₂C(Me)₂OH] [C(Me)₂CH₂OH], PenN[CH₂C(═O)OH]₂, HexN[CH₂CH₂OH]₂, HexN[CH₂CH(Me)OH]₂, HexN [CH₂CH(Me)OH] [CH(Me)CH₂OH], HexN [CHC(Me)₂OH]₂, HexN [CH₂C(Me)₂OH] [C(Me)₂CH₂OH], HexN[CH₂C(═O)OH]₂, OctN[CH₂CH₂OH]₂, OctN[CH₂CH(Me)OH]₂, OctN[CH₂CH(Me)OH] [CH(Me)CH₂OH], OctN [CH₂C(Me)₂OH]₂, OctN[CH₂C(Me)₂OH][C(Me)₂CH₂OH], OctN[CH₂C(═O)OH]₂, where Pr, Bu, Pen, Hex and Oct may be all isomeric propyl, butyl, pentyl and octyl radicals, PhN[CH₂CH₂OH]₂, PhN[CH₂CH(Me)OH]₂, PhN[CH₂CH(Me)OH] [CH(Me)CH₂OH], PhN [CH₂C(Me)₂OH]₂, PhN[CH₂C(Me)₂OH] [C(Me)₂CH₂OH] or PhN [CH₂C(═O)OH]₂.

The at least one amino alcohol is especially preferably selected from the group consisting of N-cyclopentyldiethanolamine, N-cyclohexyldiethanolamine, N-cyclopentyldiisopropanolamine, N-cyclohexyldiisopropanolamine, N-butyldiethanolamine, N-butyldiisopropanolamine, N-methyl-N,N-bis(2-hydroxybutyl)amine, N-butyl-N,N-bis(2-hydroxybutyl)amine, N-cyclohexyl-N,N-bis(2-hydroxybutyl)amine and mixtures thereof.

Methods for preparing the amino alcohols are known per se to those skilled in the art, for example by reacting the corresponding amines with oxiranes.

In the context of the present invention, “providing” generally means that the at least one amino alcohol is initially charged in such a way that the reaction envisaged in step (B) of the method according to the invention can take place. In one embodiment of the method according to the invention, the at least one amino alcohol in step (A) is initially charged in neat form, i.e. without solvent. In a preferred embodiment of the method according to the invention, the at least one amino alcohol in step (A) is provided dissolved in at least one solvent.

In general, the at least one amino alcohol may be provided without a solvent or in any solvent or solvent mixture that appears suitable to the person skilled in the art. The at least one amino alcohol in step (A) is preferably provided dissolved in at least one protic solvent. In the context of the present invention, “protic solvent” means that the solvent used has at least one proton that can be split off by a base, in particular in the form of at least one hydroxyl function.

According to the invention, preferred protic solvents are therefore alcohols, more preferably primary, secondary or tertiary alcohols. The at least one amino alcohol in step (A) of the method according to the invention is therefore preferably provided dissolved in an alcohol. The solvent used according to the invention is preferably selected from the group consisting of methanol, ethanol, propanol, in particular isopropanol, and mixtures thereof. In accordance with the invention, very particular preference is given to using methanol.

The present invention therefore preferably relates to the method according to the invention, wherein the at least one protic solvent is selected from the group consisting of alcohols, preferably methanol, ethanol, propanol, in particular isopropanol, and mixtures thereof.

In step (A) of the method according to the invention, the at least one amino alcohol may be present in at least one protic solvent at any concentration that appears suitable to the person skilled in the art. The concentration of the at least one amino alcohol in step (A) of the method according to the invention in the at least one protic solvent is preferably from 10 to 99% by weight. In step (A) of the method according to the invention, the at least one amino alcohol may also be added without a solvent, preferably a protic solvent, i.e. in neat form.

In step (A) of the method according to the invention, the at least one amino alcohol may generally be provided in any vessel or reactor known to those skilled in the art. Examples of suitable vessels or reactors are stirred reactors, circulation reactors, plug-flow reactors with internals, for example static mixers, for mixing or metered addition of the components, for example nozzles, membranes.

Step (A) of the method according to the invention can generally be carried out at any temperature that seems suitable to the person skilled in the art. Step (A) of the method according to the invention is preferably conducted at a temperature of −20 to 150° C., more preferably 0 to 30° C.

Step (A) of the method according to the invention can generally be carried out at any pressure that seems suitable to the person skilled in the art. Step (A) of the method according to the invention is preferably conducted at a pressure of 0 to 10 bar (a), more preferably at 0.1 to 1 bar (a), especially at atmospheric pressure.

Step (A) of the method according to the invention may be carried out under protective gas, for example nitrogen or argon. Step (A) of the method according to the invention is preferably carried out under protective gas.

In general, after step (A) and before step (B) of the method according to the invention, further process steps known to those skilled in the art may be carried out, for example temperature control, filtration or conditioning of the solution, for example inertizing, drying, activation by third components, stabilization. In a preferred embodiment according to the invention, step (A) is followed by step (B) without further intermediate steps.

Step (B) of the method according to the invention comprises adding at least one basic compound to the at least one amino alcohol provided in step (A) in order to obtain at least one corresponding amino alkoxide.

In step (B) of the method according to the invention, it is generally possible to use any at least one basic compound which is known to the person skilled in the art and which is capable of converting the at least one amino alcohol present to the corresponding amino alkoxide. According to the invention, preference is given to using amino alcohols having at least two hydroxyl functions. According to the invention, at least one basic compound which is able to abstract the protons from the at least two hydroxyl functions is therefore preferably used in step (B).

The at least one basic compound used in step (B) of the method according to the invention is preferably selected from the group consisting of ammonia, in aqueous and/or alcoholic solution or as liquid ammonia, mono-, di- or trivalent salts of alcohols, alkali metal or alkaline earth metal hydroxides, ammonium, alkali metal or alkaline earth metal carbonates and mixtures thereof.

In a preferred embodiment of the present invention, at least one mono-, di- or trivalent salt of at least one alcohol is used as the at least one basic compound. Preferred monovalent salts are alkali metal salts of at least one alcohol, so-called alkali metal alkoxides. Preferred alkali metals are lithium, sodium, potassium, cesium, or mixtures thereof. According to the invention, preferred divalent salts are alkaline earth metal salts of at least one alcohol, so-called alkaline earth metal alkoxides. Preferred alkaline earth metals are magnesium, calcium, barium or mixtures thereof. Alkoxides suitable according to the invention are preferably based on methanol, ethanol or mixtures thereof. According to the invention, it is therefore particularly preferable to use alkali metal methoxides, in particular sodium methoxide, as the at least one basic compound.

The present invention preferably relates to the method according to the invention wherein the at least one basic compound is ammonia, an alkali metal or alkaline earth metal salt of the anion of the at least one protic solvent, preferably a sodium alkoxide, particularly preferably selected from the group consisting of sodium methoxide, sodium ethoxide, sodium propoxide and mixtures thereof.

The at least one basic compound used in step (B) of the method according to the invention is preferably used in an amount which is sufficient to remove the protons present in the at least one amino alcohol. The molar ratio of the basic group present in the at least one basic compound to the proton present in the at least one amino alcohol is therefore preferably from 0.8 to 1.2, particularly preferably from 0.9 to 1.1, particularly preferably from 0.95 to 1.05.

Methods for preparing the metal alkoxides preferred according to the invention are known per se to those skilled in the art, for example by reacting elemental alkali metal or alkaline earth metal, alkali metal or alkaline earth metal mixtures or alloys containing alkali metal or alkaline earth metal, such as amalgams, with the corresponding alcohol with simultaneous formation of hydrogen.

In a further preferred embodiment of the present invention, the at least one basic compound used in step (B) of the method according to the invention is added in at least one solvent, preferably in at least one protic solvent.

According to the invention, preferred protic solvents in step (B) are alcohols, more preferably primary, secondary or tertiary alcohols. The at least one basic compound in step (B) of the method according to the invention is therefore preferably provided dissolved in an alcohol. The solvent used according to the invention is preferably selected from the group consisting of methanol, ethanol, propanol, in particular isopropanol, and mixtures thereof. In accordance with the invention, very particular preference is given to using methanol.

According to the invention, the at least one basic compound is preferably used dissolved in the same solvent in which the at least one amino alcohol is also provided in step (A) of the method according to the invention. Very particular preference is given to using methanol in step (A) and in step (B).

In step (B) of the method according to the invention, the at least one basic compound may be present in at least one solvent or solvent mixture, preferably in a protic solvent, in any concentration that seems suitable to the person skilled in the art. The concentration of the at least one basic compound in the at least one solvent or solvent mixture is preferably 0.1 to 100% by weight, particularly preferably 0.1 to 35% by weight.

The at least one basic compound can generally be added in step (B) of the method according to the invention in any vessel or reactor known to the person skilled in the art. The addition according to step (B) of the method according to the invention preferably takes place in the vessel or reactor in which step (A) took place. The addition per se can be carried out using devices known to those skilled in the art, for example dropping funnels.

Step (B) of the method according to the invention can generally be carried out at any temperature which seems suitable to the person skilled in the art. Step (B) of the method according to the invention is preferably carried out at a temperature of −30 to 60° C., particularly preferably −20 to 30° C.

Step (B) of the method according to the invention can generally be carried out at any pressure which appears suitable to the person skilled in the art. Step (B) of the method according to the invention is preferably conducted at a pressure of from 0 to 2 bar (a), particularly preferably at from 0.5 to 1 bar (a).

Step (B) of the method according to the invention can be carried out under protective gas, for example nitrogen or argon. Step (B) of the method according to the invention is preferably carried out under protective gas.

After step (B) of the method according to the invention, the at least one amino alcohol provided in step (A) is present in deprotonated form, preferably fully deprotonated form, i.e. as an amino alkoxide.

In general, further process steps known to those skilled in the art can be carried out after step (B) and before step (C) of the method according to the invention, for example concentration, conditioning, for example removal of dissolved inert gases, activation of components used, filtrations and/or decolorizations. In a preferred embodiment according to the invention, step (B) is followed by step (C) without further intermediate steps, with inertization optionally being carried out.

Step (C) of the method according to the invention comprises the addition of at least one metal halide to the mixture obtained in step (C) in order to obtain the corresponding at least one metal amino alkoxide, wherein in step (C) the at least one metal halide is added as a solution in a protic solvent at a concentration of 0.1 to 5.0% by weight.

In general, in step (C) of the method according to the invention, it is possible to use any metal halide which appears suitable to those skilled in the art and which reacts with the at least one amino alkoxide present in the solution from step (B) to give the at least one metal amino alkoxide desired according to the invention.

It is essential to the invention that in step (C) the at least one metal halide is added as a solution in a protic solvent at a concentration of 2.0 to 35.0% by weight, preferably 5.0 to 15.0% by weight, based in each case on the added solution. The pressure applied is from 0 to 5 bar (a), preferably from 0.2 to 0.95 bar (a), particularly preferably from 0.4 to 0.8 bar (a).

The at least one metal present in the metal amino alkoxide according to the invention is preferably selected from the group consisting of tin, in particular tin(IV), titanium, in particular titanium(IV), bismuth, in particular Bi(III), and mixtures thereof.

The present invention therefore preferably relates to the method according to the invention, wherein the at least one metal is selected from the group consisting of tin, in particular tin(IV), titanium, in particular titanium(IV), bismuth, in particular Bi(III), and mixtures thereof.

The at least one metal halide used in step (C) of the method according to the invention is particularly preferably selected from the group consisting of tin(IV) chloride, titanium(IV) chloride and mixtures thereof.

The at least one metal halide used in step (C) of the method according to the invention is preferably used in an amount such that all of the at least one amino alkoxide present from step (B) can be reacted. The molar ratio of the deprotonated hydroxyl function present in the at least one amino alkoxide to the valency of the at least one metal cation present, i.e. preferably +IV, is therefore preferably from 0.8 to 1.2, particularly preferably from 0.95 to 1.05.

Step (C) of the method according to the invention can generally be carried out at any temperature which seems suitable to the person skilled in the art. Step (C) of the method according to the invention is preferably carried out at a temperature of −30 to 60° C., particularly preferably −20 to 30° C.

Step (C) of the method according to the invention can generally be carried out at any pressure which appears suitable to the person skilled in the art. Step (C) of the method according to the invention is preferably conducted at a pressure of from 0 to 2 bar (a), particularly preferably at from 0.5 to 1 bar (a).

Step (C) of the method according to the invention can be carried out under protective gas, for example nitrogen or argon. Step (C) of the method according to the invention is preferably carried out under protective gas.

After step (C) of the method according to the invention, a solution is obtained of the at least one corresponding metal amino alkoxide in the at least one protic solvent.

Step (C) of the process according to the invention can be followed by further process steps known per se to those skilled in the art, for example work-up and/or purification. In particular, any solids present can be filtered off. Furthermore, the solution obtained may be freed from the at least one protic solvent, especially by distillation. The residue thus obtained can then be taken up with a suitable solvent.

According to the invention, this solvent may be selected from the group consisting of esters, preferably butyl acetate, especially n-butyl acetate, ethyl acetate, ethylene glycol diacetate, 2-methoxypropyl acetate or mixtures thereof, aromatic solvents, preferably benzene, toluene, xylenes or mixtures thereof, lactones, preferably butyrolactone or mixtures thereof, carbonates, preferably diethyl carbonate, propylene carbonate, ethylene carbonate or mixtures thereof.

The present invention furthermore also relates to a solution comprising at least one metal alkoxide, prepared by the method according to the invention.

The at least one solvent present in the solution according to the invention may be a protic solvent selected from the group consisting of methanol, ethanol, propanol, in particular isopropanol, and mixtures thereof.

In addition, the at least one solvent present in the solution according to the invention may be selected from the group consisting of esters, preferably butyl acetate, especially n-butyl acetate, ethyl acetate, ethylene glycol diacetate, 2-methoxypropyl acetate or mixtures thereof, aromatic solvents, preferably benzene, toluene, xylenes or mixtures thereof, lactones, preferably butyrolactone or mixtures thereof, carbonates, preferably diethyl carbonate, propylene carbonate, ethylene carbonate or mixtures thereof.

The solution according to the invention preferably has a Hazen colour number, measured spectrophotometrically in accordance with DIN EN ISO 6271-2:2005-03, of less than 500 APHA, preferably less than 200 APHA, particularly preferably less than 100 APHA, especially preferably less than 80 APHA, particularly less than 60 APHA. The Hazen colour number, measured spectrophotometrically in accordance with DIN EN ISO 6271-2:2005-03 is in this case preferably at least 1 APHA.

The present invention also relates to the use of the solution according to the invention for producing a composition comprising the at least one metal amino alkoxide, at least one solvent selected from the group consisting of esters, preferably butyl acetate, especially n-butyl acetate, ethyl acetate, ethylene glycol diacetate, 2-methoxypropyl acetate or mixtures thereof, aromatic solvents, preferably benzene, toluene, xylenes or mixtures thereof, lactones, preferably butyrolactone or mixtures thereof, carbonates, preferably diethyl carbonate, propylene carbonate, ethylene carbonate or mixtures thereof, and mixtures thereof, at least one polyisocyanate and at least one NCO-reactive compound.

Furthermore, the present invention relates to the composition comprising at least one polyisocyanate, at least one NCO-reactive compound and a solution according to the invention.

The present invention relates in particular to a composition comprising the at least one metal amino alkoxide, at least one solvent selected from the group consisting of esters, preferably butyl acetate, especially n-butyl acetate, ethyl acetate, ethylene glycol diacetate, 2-methoxypropyl acetate or mixtures thereof, aromatic solvents, preferably benzene, toluene, xylenes or mixtures thereof, lactones, preferably butyrolactone or mixtures thereof, carbonates, preferably diethyl carbonate, propylene carbonate, ethylene carbonate or mixtures thereof, and mixtures thereof, and at least one polyisocyanate.

This composition according to the invention is distinguished from similar compositions of the prior art in that it is storage-stable over a long period of time and does not become discolored. The present invention therefore preferably relates to the composition according to the invention, wherein after storage for at least 3 months, preferably for at least 6 months, at a maximum temperature of 40° C., said composition has a Hazen color number, measured spectrophotometrically according to DIN EN ISO 6271-2:2005-03, of less than 150 APHA, preferably less than 90 APHA, in particular less than 80 APHA. The Hazen colour number, measured spectrophotometrically in accordance with DIN EN ISO 6271-2:2005-03 is in this case preferably at least 1 APHA.

The present invention also relates to the use of the composition according to the invention for producing single-layered or multi-layered coating systems.

The present invention also relates to a process for single-layered or multi-layered coating of a substrate with a coating system by applying a composition according to the invention to the substrate.

The present invention also relates to the substrate, coated with a single-layered or multi-layered coating system, comprising a composition according to the invention and/or obtainable by the method according to the invention, wherein in particular the substrate is a chassis, preferably of a vehicle, especially of a land vehicle, aircraft or watercraft, or a part thereof.

Corresponding single-layer or multi-layer coating systems, compositions and methods for producing the corresponding coatings are described, for example, in WO 2017/182429 A1.

Suitable polyisocyanates in accordance with the invention are described, for example, in WO 2017/182429 A1 and are preferably selected from the group consisting of di- or triisocyanates, for example butane 1,4-diisocyanate, pentane 1,5-diisocyanate (pentamethylene diisocyanate, PDI), hexane 1,6-diisocyanate (hexamethylene diisocyanate, HDI), 4-isocyanatomethyloctane 1,8-diisocyanate (triisocyanatononane, TIN), 4,4′-methylenebis(cyclohexyl isocyanate) (H₁₂MDI), 3,5,5-trimethyl-1-isocyanato-3-isocyanatomethylcyclohexane (isophorone diisocyanate, IPDI), 1,3- and 1,4-bis(isocyanatomethyl)cyclohexane (H₆XDI), naphthalene 1,5-diisocyanate, diisocyanatodiphenylmethane (2,2′-, 2,4′- and 4,4′-MDI or mixtures thereof), diisocyanatomethylbenzene (toluylene 2,4- and 2,6-diisocyanate, TDI) and technical grade mixtures of the two isomers, and also 1,3- and/or 1,4-bis(isocyanatomethyl)benzene (XDI), 3,3′-dimethyl-4,4′-biphenyl diisocyanate (TODI), paraphenylene 1,4-diisocyanate (PPDI) and cyclohexyl diisocyanate (CHDI) and the oligomers of higher molecular weight that are obtainable individually or in a mixture from the above and have biuret, uretdione, isocyanurate, iminooxadiazinedione, allophanate, urethane and carbodiimide/uretonimine structural units, and mixtures thereof.

Preference is given to the use of polyisocyanates based on aliphatic and cycloaliphatic diisocyanates. The present invention therefore relates in addition to the composition according to the invention comprising at least one polyisocyanate, at least one NCO-reactive compound and a solution according to the invention, i.e. the solution B according to the invention.

Suitable NCO-reactive compounds in accordance with the invention are described, for example, in WO 2017/182429 A1 and in particular are selected from the group consisting of low molecular weight diols, e.g. 1,2-ethanediol, 1,3- or 1,2-propanediol, 1,4-butanediol, triols, e.g. glycerol, trimethylolpropane, tetraols e.g. pentaerythritol, short-chain polyamines, polyhydroxy compounds such as polyether polyols, polyester polyols, polyurethane polyols, polysiloxane polyols, polycarbonate polyols, polyether polyamines, polybutadiene polyols, polyacrylate polyols, polymethacrylate polyols, mixed polymers thereof, and mixtures thereof.

The polyhydroxyl compounds preferably have mass-average molecular weights Mw>500 daltons, measured by means of gel permeation chromatography (GPC) against a polystyrene standard, more preferably between 800 and 100 000 daltons, especially between 1000 and 50 000 daltons.

The polyhydroxyl compounds preferably have an OH number of 30 to 400 mg KOH/g, especially between 100 and 300 KOH/g. The hydroxyl number (OH number) indicates how many mg of potassium hydroxide are equivalent to the amount of acetic acid bound by 1 g of substance in the acetylation. In the determination, the sample is boiled with acetic anhydride/pyridine, and the acid formed is titrated with potassium hydroxide solution (DIN 53240-2).

The glass transition temperatures, measured with the aid of DSC measurements according to DIN EN ISO 1 1357-2, of the polyhydroxyl compounds are preferably between −150 and 100° C., more preferably between −120° C. and 80° C.

### EXAMPLES

Methods and Materials:

The Hazen colour number was measured by spectrophotometry according to DIN EN ISO 6271-2:2005-03 with a LICO 400 spectrophotometer from Lange, Germany.

Unless otherwise stated, the compounds used were obtained from chemical retailers, for example Sigma-Aldrich, Merck KGaA, Darmstadt, Germany or BASF SE, Ludwigshafen, Germany, and were each present in a purity of more than 99% by weight.

The identity and purity of each of the newly synthesized compounds was unequivocally determined from the results of NMR spectroscopy, gas chromatography and mass spectrometry investigations.

Preparation methods for the commercially unavailable N-alkyldialkanolamines used as ligands for the catalysts in Examples a), b), c), d), f), g), h) and i)

General Preparation Method

The particular amine specified in Table 1 below was placed in a 2 l laboratory autoclave under a nitrogen atmosphere. The amount weighed in was such that it was in a molar ratio of 1:2 with the epoxides metered in, resulting in a theoretical total yield of 1500 g in each case. Oxygen was removed at room temperature by four cycles of pressurization of the autoclave with nitrogen to 3 bar (a) and subsequent release of the positive pressure to standard pressure. After the autoclave had been closed, its contents were heated to 90° C. (reaction temperature) with stirring (800 rpm, cross-bar stirrer). After reaching the reaction temperature and a reaction start pressure depending on the respective amine, the epoxide specified in the table was metered into the headspace of the autoclave with continuous stirring in such a way that a pressure of 5 bar was not exceeded. In relation to the amount of amine weighed out, twice the amount of epoxide was used in each case. After the end of the epoxide metered addition, a post-reaction phase followed. This post-reaction phase was considered to be completed when the pressure drop was below 20 mbar/h. The product was subsequently devolatilized at 90° C. under reduced pressure (30 mbar) over a period of 30 minutes.

TABLE 1
Reactants used

| Example | Amine used | Epoxide used |
|---|---|---|
| a) | Cyclopentylamine | Ethylene oxide* |
| b) | Cyclohexylamine | Ethylene oxide* |
| c) | Cyclopentylamine | Methyloxirane (propylene oxide) |
| d) | Cyclohexylamine | Methyloxirane (propylene oxide) |
| e) | n-Butylamine | Methyloxirane (propylene oxide) |
| g) | Methylamine** | Ethyloxirane (1-Butene oxide) |
| h) | n-Butylamine | Ethyloxirane (1-Butene oxide) |
| i) | Cyclohexylamine | Ethyloxirane (1-Butene oxide) |

*In the experiments in which ethylene oxide (oxirane) was metered in, a nitrogen inlet pressure of 2.8 bar was set before the start of epoxide metered addition.
**Deviating from the procedure given in the general preparation method, a 40% aqueous solution of methylamine was used here. The reaction temperature was 75° C. After the end of the ethyloxirane metered addition, the water was removed in vacuo at 80° C. over a period of 3 hours.

The alkanolamines thus obtained were then purified by rectification in vacuo and obtained as colorless liquids or oils. Refractive indices and boiling points of the compounds obtained are given in Table 2.

TABLE 2
Refractive indices and boiling points of the amino alcohols

| | | | Kp @ 0.1 |
|---|---|---|---|
| Example | Amino alcohol | n_{D}²⁰ | mbar [° C.] |
| a) | N-Cyclopentyldiethanolamine | 1.4961 | 110 |
| b) | N-Cyclohexyldiethanolamine | 1.4935 | 120 |
| c) | N-Cyclopentyldiisopropanolamine | 1.4785 | 117 |
| d) | N-Cyclohexyldiisopropanolamine | 1.4804 | 100 |
| f) | N-Butyldiisopropanolamine | 1.4518 | 73 |
| g) | N-Methyl-N,N- | 1.4531 | 66 |
| | bis(2-hydroxybutyl)amine | | |
| h) | N-Butyl-N,N- | 1.4546 | 97 |
| | bis(2-hydroxybutyl)amine | | |
| i) | N-Cyclohexyl-N,N- | 1.4798 | 127 |
| | bis(2-hydroxybutyl)amine | | |

### Examples 1a to 1i (Non-Inventive, SnCl₄ Undiluted)

A 1 l four-necked flask equipped with a jacketed coil condenser (brine-cooled), PTFE-jacketed internal thermometer (PT 100), mechanical PTFE-jacketed stirrer and pressure-equalizing dropping funnel was respectively initially charged under an inert gas atmosphere (dry nitrogen) with

### a) 133.0 g of N-cyclopentyldiethanolamine,

### b) 143.8 g of N-cyclohexyldiethanolamine,

### c) 154.6 g of N-cyclopentyldiisopropanolamine,

### d) 165.3 g of N-cyclohexyldiisopropanolamine,

### e) 123.8 g of N-butyldiethanolamine,

### f) 145.3 g of N-butyldiisopropanolamine,

g) 134.6 g of N-methyl-N,N-bis(2-hydroxybutyl)amine,
h) 166.9 g of N-butyl-N,N-bis(2-hydroxybutyl)amine or
i) 186.9 g of N-cyclohexyl-N,N-bis(2-hydroxybutyl)amine
and 276.5 g of a 30% methanolic sodium methoxide solution was added in each case. The reactor contents were uniformly adjusted to ca. 600 ml by adding further methanol. Then, with stirring and external brine cooling (internal temperature 0 to 5° C.), 100 g of freshly distilled tin tetrachloride were slowly added dropwise in each case using a pressure-equalizing dropping funnel.

There was an onset of significant mist binding in the reactor and solids separation in the liquid. Irrespective of the speed of dropwise addition and stirring, caking formed on the dropping funnel and sometimes also above the liquid surface in a manner that was difficult to control, and even vigorous stirring and the application of a vacuum until the reaction mixture boiled could not break it up reproducibly and completely and convert it to the liquid phase.

After filtration, washing of the precipitates with 3 times ca. 100 ml of fresh methanol, concentration in vacuo to constant pressure at a maximum product temperature of 30° C. and ca. 0.1 mbar, the product was purged with nitrogen and each time taken up with ca. 500 ml of n-butyl acetate, undissolved components were filtered off and the product was distilled on an effective column at gradually reduced pressure until pure n-butyl acetate was present at the head, i.e. methanol was no longer present in the receiver.

The clear, slightly discolored solutions remaining as distillation bottoms (Hazen color number <200 Apha in each case) were adjusted to a uniform 1.0% by weight tin content—determined in each case by means of X-ray fluorescence analysis—and used to produce the products listed in example series 3-1.

Experiments 1a to 1 i were repeated, with the difference that 99.5 g (experiments 1a-1 to 1i-1) or 100.5 g (experiments 1a-2 to 1i-2) of freshly distilled tin tetrachloride were added. The course of the reaction was optically identical; in terms of analysis (NMR, elemental analysis, color determination) there were practically no apparent differences in the respective 3 products obtained of the same chemical structure. These solutions were also used to prepare the products listed in example series 3-1.

### Examples 2a to 2i (Inventive, SnCl₄ Diluted)

A 1 l four-necked flask equipped with a jacketed coil condenser (brine-cooled), PTFE-jacketed internal thermometer (PT 100), mechanical PTFE-jacketed stirrer and pressure-equalizing dropping funnel was respectively initially charged under an inert gas atmosphere (dry nitrogen) with

### a) 13.3 g of N-cyclopentyldiethanolamine,

### b) 14.4 g of N-cyclohexyldiethanolamine,

### c) 15.5 g of N-cyclopentyldiisopropanolamine,

### d) 16.5 g of N-cyclohexyldiisopropanolamine,

### e) 12.4 g of N-butyldiethanolamine,

### f) 14.5 g of N-butyldiisopropanolamine,

g) 13.5 g of N-methyl-N,N-bis(2-hydroxybutyl)amine,
h) 16.7 g of N-butyl-N,N-bis(2-hydroxybutyl)amine or
i) 18.7 g of N-cyclohexyl-N,N-bis(2-hydroxybutyl)amine
and 27.7 g of a 30% methanolic sodium methoxide solution was added in each case. The reactor contents were uniformly adjusted to ca. 100 ml by adding further methanol.

Then, with stirring and external brine cooling (internal temperature 0 to 5° C.), in each case 1000 g of a virtually colourless, clear, liquid mixture consisting of 100 g of freshly distilled tin tetrachloride and 900 g of methanol (hereinafter: 10% methanolic SnCl₄ solution) were slowly added dropwise using a pressure-equalizing dropping funnel.

There was no binding of mist in the reactor and the separation of solids in the liquid appeared to be similar to that in experiment series 2. In no case was the formation of caking observed on the dropping funnel or above the liquid surface.

The clear, virtually colorless solutions remaining after work-up as described under 2 (Hazen color number <100 Apha in each case) were adjusted to a uniform 1.0% by weight tin content—determined in each case by means of X-ray fluorescence analysis—and used to produce the products listed in example series 4-2.

Experiments 2a to 2i were repeated, with the difference that 995 g (experiments 2a-1 to 2i-1) or 1005 g (experiments 2a-2 to 2i-2) of the 10% methanolic SnCl₄ solution were added. The course of the reaction was optically identical; in terms of analysis (NMR, elemental analysis, color determination) there were practically no apparent differences in the respective 3 products obtained of the same chemical structure. These solutions were also used to prepare the products listed in example series 3-2.

### Example Series 3

(Preparation of Mixtures of Tin(IV) Amino Alkoxides and Polyisocyanates; Comparative Experiments: Series 3-1, Experiments According to the Invention: Series 3-2)

In each case 90 g of an HDI polyisocyanate (isocyanurate type, product Desmodur N 3300 from Covestro AG) were mixed with 6.75 g each of the solutions obtained in Example 1 or Example 2 and then made up to a uniform 100 g in each case by adding n-butyl acetate.

The mixtures were homogenized at 50° C. for one hour and then stored at 40° C. After visually observing a clear change in the habitus of the sample, the colour, viscosity and NCO content were determined.

For 80% of the products based on the tin(IV) amino alkoxide solutions obtained in the comparative experiment series 1a to 1i, 1a-1 to 1i-1 and 1a-2 to 1i-2, this maximum storage time was less than 2 months. None of the samples were visually impeccable for longer than 4 months, i.e. the Hazen color number was above 150 Apha in each case.

In a non-specific manner, strong color deepening and viscosity increases sometimes occurred in parallel, but mostly only color deepening (Hazen color number >>500 Apha) towards deep yellow and orange hues were observed.

In contrast, surprisingly, no such effects occurred in series 3-2 according to the invention when using the tin(IV) amino alkoxide solutions obtained by the method according to the invention according to Examples 2a to 2i/2a-1 to 2i-1 and 2a-2 to 2i-2. None of the samples were remarkable in terms of color or viscosity/NCO content after storage for 4 months.

1. A method for preparing at least one metal amino alkoxide, comprising:
(A) providing at least one amino alcohol,
(B) adding at least one basic compound to the at least one amino alcohol provided in step (A) to obtain at least one corresponding amino alkoxide, and
(C) adding at least one metal halide to the mixture obtained in step (B) to obtain the corresponding at least one metal amino alkoxide,
characterized in that in step (C) the at least one metal halide is added as a solution in a protic solvent at a concentration of 2.0 to 35.0% by weight.

2. The method as claimed in claim 1, characterized in that the metal is selected from the group consisting of tin, titanium, bismuth, and mixtures thereof.

3. The method as claimed in claim 1, characterized in that the at least one basic compound added in step (B) a solution in a protic solvent.

4. The method as claimed claim 1, characterized in that the at least one amino alcohol provided in step (A) of the method conforms to the general formula (I) or (II)
HO—X-D-Y—OH (I)
HO—X-D-Z-D-Y—OH (II)
wherein
D is —O—, —S— or —N(R1)-,
wherein at least one D present is —N(R1)-,
wherein R1 is a saturated or unsaturated, linear or branched, aliphatic or cycloaliphatic radical or an optionally substituted aromatic or araliphatic radical having up to 20 carbon atoms, which may optionally comprise heteroatoms from the group of oxygen, sulfur and nitrogen, or is hydrogen,
X, Y and Z are identical or different radicals selected from alkylene radicals of the formulae —C(R2)(R3)-, —C(R2)(R3)-C(R4)(R5)- or —C(R2)(R3)-C(R4)(R5)-C(R6)(R7)- or ortho-arylene radicals of the formulae 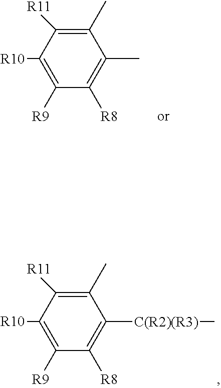
wherein R2 to R11 are each independently saturated or unsaturated, linear or branched, aliphatic or cycloaliphatic radicals or optionally substituted aromatic or araliphatic radicals having up to 20 carbon atoms, which may optionally comprise heteroatoms from the group of oxygen, sulfur and nitrogen, or are hydrogen.

5. The method as claimed in claim 1, characterized in that the at least one amino alcohol is selected from the group consisting of N-cyclopentyldiethanolamine, N-cyclohexyldiethanolamine, N-cyclopentyldiisopropanolamine, N-cyclohexyldiisopropanolamine, N-butyldiethanolamine, N-butyldiisopropanolamine, N-methyl-N,N-bis(2-hydroxybutyl)amine, N-butyl-N,N-bis(2-hydroxybutyl)amine, N-cyclohexyl-N,N-bis(2-hydroxybutyl)amine, and mixtures thereof.

6. The method as claimed in claim 1, characterized in that the at least one basic compound is selected from the group consisting of ammonia, an alkali metal, and an alkaline earth metal salt of the anion of the at least one protic solvent.

7. The method as claimed in claim 1, characterized in that at least one protic solvent is selected from the group consisting of alcohols.

8. A solution comprising at least one metal alkoxide, prepared by the method according to claim 1.

9. (canceled)

10. A composition comprising at least one polyisocyanate, at least one NCO-reactive compound and a solution as claimed in claim 8.

11. A composition comprising at least one metal amino alkoxide, at least one solvent selected from the group consisting of esters, aromatic solvents, lactones, carbonates and mixtures thereof, and at least one polyisocyanate.

12. The composition as claimed in claim 11, characterized in that after storage for at least 3 months, at a maximum temperature of 40° C., said composition has a Hazen color number, measured spectrophotometrically according to DIN EN ISO 6271-2:2005-03, of less than 150 APHA.

13. (canceled)

14. A method for single-layered or multi-layered coating of a substrate with a coating system by applying a composition as claimed in claim 10 to the substrate.

15. A substrate coated with a single-layered or multi-layered coating system comprising a composition as claimed in claim 10 wherein the substrate is a chassis of a land vehicle, aircraft or watercraft, or a part thereof.

16. A substrate coated with a single-layered or multi-layered coating system comprising a composition obtained by the method as claimed in claim 14, wherein the substrate is a chassis of a land vehicle, aircraft or watercraft, or a part thereof.
